(19) United States
(12) Patent Application Publication
(10) Pub. No.: US 2020/0002337 A1
(43) Pub. Date: Jan. 2, 2020

(54) DIHYDROPYRIDOPHTHALAZINONE COMPOUNDS AS INHIBITORS OF POLY (ADP-RIBOSE) POLYMERASE (PARP) FOR TREATMENT OF DISEASES AND METHOD OF USE THEREOF

(71) Applicant: SUZHOU KINTOR PHARMACEUTICALS, INC., Suzhou, Jiangsu (CN)

(72) Inventors: Chuangxing GUO, San Diego, CA (US); Youzhi TONG, Little Neck, NY (US)

(73) Assignee: SUZHOU KINTOR PHARMACEUTICALS, INC., Suzhou, Jiangsu (CN)

(21) Appl. No.: 16/487,919

(22) PCT Filed: Feb. 9, 2018
(86) PCT No.: PCT/CN2018/075907

§ 371 Date: Aug. 22, 2019

Related U.S. Application Data
(60) Provisional application No. 62/463,609, filed on Feb. 25, 2017.

(51) Int. Cl. C07D 471/06; A61P 35/00

(52) U.S. Cl. CPC C07D 471/06; A61P 35/00

(57) ABSTRACT

The present invention provides novel dihydropyridophthalazinone compounds of Formula (I) as PARP inhibitors, and their pharmaceutically acceptable salts, solvates, hydrates, prodrugs and metabolites thereof, the preparation thereof, and the use of such compounds to treat DNA repair dysregulation diseases and conditions such as cancer. The present provides therapeutic applications for the treatment of stroke, myocardial infarction, neurodegenerative diseases, ovarian cancer, breast cancer, prostate cancer, lung cancer, colorectal cancer, and melanoma.

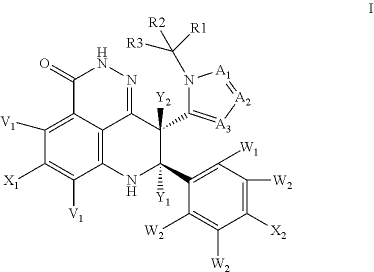

### CROSS REFERENCE TO RELATED APPLICATIONS

This application claims priority benefit of U.S. Patent Application No. U.S. 62463609 filed Feb. 25, 2017, the contents of which are incorporated herein by reference in their entirety.

### FIELD OF THE INVENTION

The present invention is directed to novel dihydropyridophthalazinone compounds as PARP inhibitors, and their pharmaceutically acceptable salts, solvates, hydrates, prodrugs and metabolites thereof, the preparation thereof, and the use of such compounds to treat DNA repair dysregulation diseases and conditions such as cancer.

### BACKGROUND OF THE INVENTION

DNA is damaged thousands of times during each cell cycle, and that damage must be repaired. BRCA1, BRCA2 and PALB2 are proteins that are important for the repair of double-strand DNA breaks by the error-free homologous recombinational repair, or HRR, pathway. When the gene for either protein is mutated, the change can lead to errors in DNA repair that can eventually cause breast cancer. When subjected to enough damage at one time, the altered gene can cause the death of the cells.

PARP1 is a protein that is important for repairing single-strand breaks (‘nicks’ in the DNA). If such nicks persist unrepaired until DNA is replicated (which must precede cell division), then the replication itself can cause double strand breaks to form.

Drugs that inhibit PARP1 cause multiple double strand breaks to form in this way, and in tumors with BRCA1, BRCA2 or PALB2 mutations these double strand breaks cannot be efficiently repaired, leading to the death of the cells. Normal cells that don't replicate their DNA as often as cancer cells, and that lack any mutated BRCA1 or BRCA2 still have homologous repair operating, which allows them to survive the inhibition of PARP.

Some cancer cells that lack the tumor suppressor PTEN or low in oxygen (e.g. in fast growing tumors) may be sensitive to PARP inhibitors.

### SUMMARY OF THE INVENTION

One aspect of the invention provides a compound of Formula I:

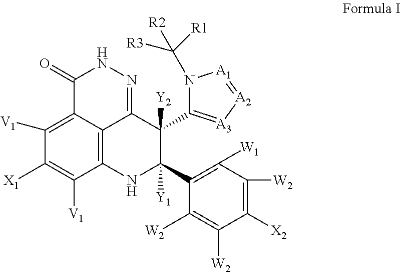

or a pharmaceutically acceptable salt, solvate, stereoisomer or tautomer thereof, wherein R₁, R₂, R₃, Y₁, Y₂, W₁, W₂, X₁, and X₂ are independently selected from H, deuterium and F, provided that R₁, R₂, R₃, Y₁, Y₂, W₁, W₂, X₁, and X₂ contain at least one deuterium; and wherein A₁, A₂, and A₃ are independently selected from N and CH. In some embodiments, X₁ is F. In other embodiments, X₂ is F. In some embodiments, X₁ and X₂ are both F. In other embodiments, A₁ is N, A₂ is CH and A₃ is N. In some embodiments, Y₁ is deuterium. In other embodiments, —CR₁R₂R₃ is —CD₃. In some embodiments, Y₂ is deuterium.

Another aspect of the invention provides a compound of Formula II:

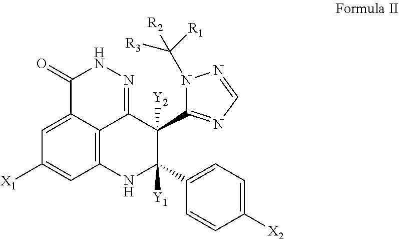

or a pharmaceutically acceptable salt, solvate, stereoisomer or tautomer thereof, wherein R₁, R₂, R₃, Y₁, Y₂, X₁, and X₂ are independently selected from H, deuterium and F, provided that R₁, R₂, R₃, Y₁, Y₂, X₁, and X₂ contain at least one deuterium. In some embodiments, X₁ is F. In other embodiments, X₂ is F. In some embodiments, X₁ and X₂ are both F. In other embodiments, Y₁ is deuterium. In some embodiments, —CR₁R₂R₃ is —CD₃. In other embodiments, Y₂ is deuterium.

Still another aspect of the invention provides a compound of Formula III:

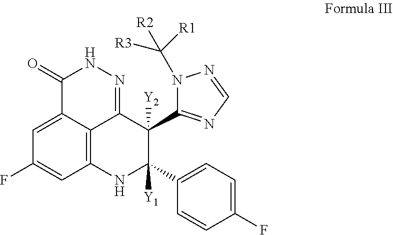

or a pharmaceutically acceptable salt, solvate, stereoisomer or tautomer thereof, wherein R₁, R₂, R₃, Y₁, and Y₂ are independently selected from H, deuterium and F, provided that R₁, R₂, R₃, Y₁, and Y₂ contain at least one deuterium. In some embodiments, Y₁ is deuterium. In other embodiments, —CR₁R₂R₃ is —CD₃.

In some embodiments, the compound is selected from the group consisting of:

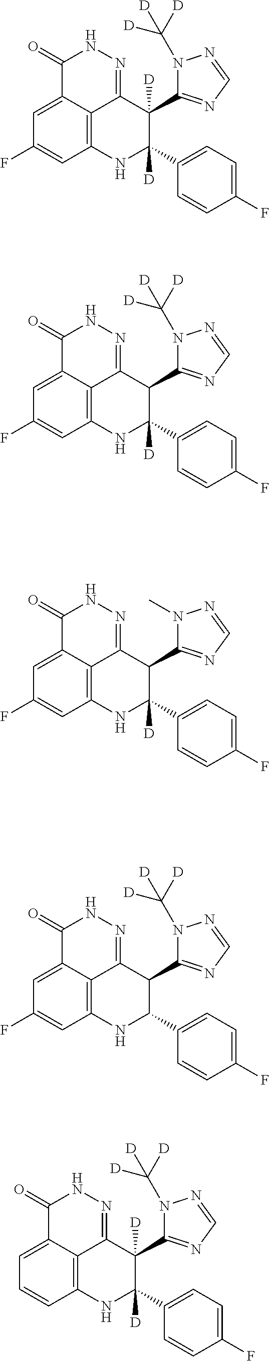 [SMILES: [2H]C([2H])([2H])n1ncnc1[C@]1([2H])c2n[nH]c(=O)c3cc(F)cc(c23)N[C@@]1([2H])c1ccc(F)cc1 ; [2H]C([2H])([2H])n1ncnc1[C@@H]1c2n[nH]c(=O)c3cc(F)cc(c23)N[C@@]1([2H])c1ccc(F)cc1 ; [2H][C@@]1(c2ccc(F)cc2)Nc2cc(F)cc3c(=O)[nH]nc(c23)[C@H]1c1ncnn1C ; [2H]C([2H])([2H])n1ncnc1[C@@H]1c2n[nH]c(=O)c3cc(F)cc(c23)N[C@H]1c1ccc(F)cc1 ; [2H]C([2H])([2H])n1ncnc1[C@]1([2H])c2n[nH]c(=O)c3cccc(c23)N[C@@]1([2H])c1ccc(F)cc1]

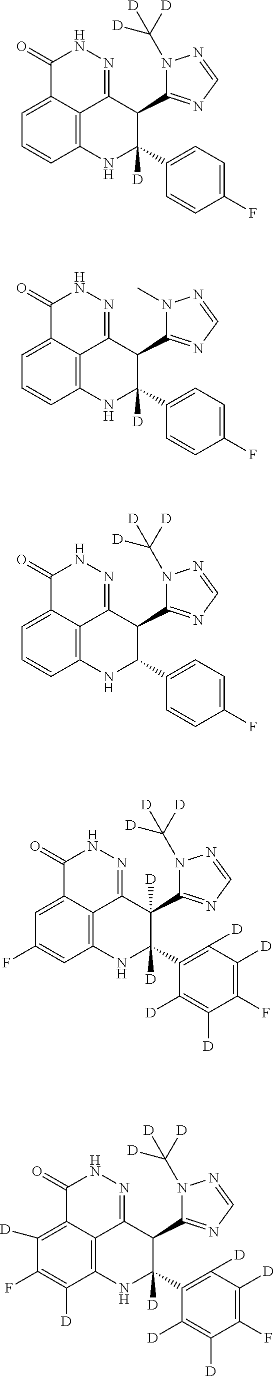 [SMILES: [2H]C([2H])([2H])n1ncnc1[C@@H]1c2n[nH]c(=O)c3cccc(c23)N[C@@]1([2H])c1ccc(F)cc1 ; [2H][C@@]1(c2ccc(F)cc2)Nc2cccc3c(=O)[nH]nc(c23)[C@H]1c1ncnn1C ; [2H]C([2H])([2H])n1ncnc1[C@@H]1c2n[nH]c(=O)c3cccc(c23)N[C@H]1c1ccc(F)cc1 ; [2H]c1c([2H])c([C@]2([2H])Nc3cc(F)cc4c(=O)[nH]nc(c34)[C@@]2([2H])c2ncnn2C([2H])([2H])[2H])c([2H])c([2H])c1F ; [2H]c1c([2H])c([C@]2([2H])Nc3c([2H])c(F)c([2H])c4c(=O)[nH]nc(c34)[C@H]2c2ncnn2C([2H])([2H])[2H])c([2H])c([2H])c1F]

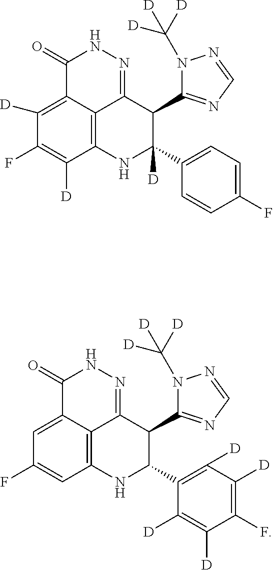 [SMILES: [2H]c1c(F)c([2H])c2c(=O)[nH]nc3c2c1N[C@@]([2H])(c1ccc(F)cc1)[C@@H]3c1ncnn1C([2H])([2H])[2H] ; [2H]c1c([2H])c([C@@H]2Nc3cc(F)cc4c(=O)[nH]nc(c34)[C@H]2c2ncnn2C([2H])([2H])[2H])c([2H])c([2H])c1F]

In some embodiments, the pharmaceutically acceptable salt is the salt prepared by adding acids to the compounds with formula such as I, II, III.

In some specific embodiments, the acids are inorganic acid or organic acid. Wherein the inorganic acids include but not limited to HCl, H₃PO₄, H₂SO₄, HNO₃, HBr, HI et al, the organic acids include but not limited to formic acid, acetic acid, CF₃COOH, propionic acid, butyric acid, oxalic acid, hexanedioic acid, malic acid, tartaric acid, semi tartaric acid, amino acids, methanesulfonic acid, benzene sulfonic acid, p-TsOH, naphthalene sulfonic acid, fumaric acid, maleic acid, succinic acid, cholic acid, desoxycholic acid, citric acid, Mucic Acid, hippuric acid, gentisic acid et al. The organic acid herein can have chiral or no chiralcenter. For the acid with stereocenters, the salt form can be pure enantiomer, racemate, or diastereomers.

Another aspect of the invention provides a pharmaceutical composition comprising a compound selected from Formulas I-III or a pharmaceutically acceptable salt thereof, and a pharmaceutically acceptable carrier.

Still another aspect of the invention provides a method for treating a disease or disorder related to PARP inhibition, comprising administering a pharmaceutical composition described above. In some embodiments, the disorder is related hyperplasia related to defective DNA repair pathways. In other embodiments, wherein the disorder is related hyperplasia related to BRCA1 and/or BRCA2 mutations. In some embodiments, the disorder is related hyperplasia.

Before proceeding with the detailed description, it is to be appreciated that the following detailed description is merely exemplary in nature and is not intended to limit the invention or the application and uses thereof. Hence, although the present disclosure is, for convenience of explanation, depicted and described as shown in certain illustrative embodiments, it will be appreciated that it can be implemented in various other types of embodiments and equivalents, and in various other systems and environments. Furthermore, there is no intention to be bound by any theory presented in the preceding background or the following detailed description.

### DETAILED DESCRIPTION OF THE INVENTION

### Definitions

Compounds are generally described herein using standard nomenclature. For compounds having asymmetric centers, it should be understood that (unless otherwise specified) all of the optical isomers and mixtures thereof are encompassed. In addition, compounds with carbon-carbon double bonds may occur in Z- and E-forms, with all isomeric forms of the compounds being included in the present invention unless otherwise specified. Where a compound exists in various tautomeric forms, a recited compound is not limited to any one specific tautomer, but rather is intended to encompass all tautomeric forms.

A “substituent” and “substituted,” as used herein, denote that a molecular moiety is covalently bonded to an atom within a molecule of interest. For example, a ring substituent may be a moiety such as a halogen, alkyl group, haloalkyl group or other group that is covalently bonded to an atom (preferably a carbon or nitrogen atom) that is a ring member. Substituents of aromatic groups are generally covalently bonded to a ring carbon atom.

The term “pharmaceutically acceptable” when used with reference to a compound of formula I is intended to refer to a form of the compound that is safe for administration to a subject. For example, a free base, a salt form, a solvate, a hydrate, a prodrug or derivative form of a compound of formula I, which has been approved for mammalian use, via oral ingestion or any other route of administration, by a governing authority or regulatory agency, such as the Food and Drug Administration (FDA) of the United States, is pharmaceutically acceptable.

Included in the compounds of formula I are the pharmaceutically acceptable salt forms of the free-base compounds. The term “pharmaceutically-acceptable salts” embraces salts, commonly used to form alkali metal salts and to form addition salts of free acids or free bases, which have been approved by a regulatory agency. Salts are formed from ionic associations, charge-charge interactions, covalent bonding, complexation, coordination, etc. The nature of the salt is not critical, provided that it is pharmaceutically acceptable.

In some embodiments, the compound(s) of formula I is used to treat a subject by administering the compound(s) as a pharmaceutical composition. To this end, the compound(s), in one embodiment, is combined with one or more pharmaceutically acceptable excipients, including carriers, diluents or adjuvants, to form a suitable composition, which is described in more detail herein.

The term “excipient”, as used herein, denotes any pharmaceutically acceptable additive, carrier, adjuvant, or other suitable ingredient, other than the active pharmaceutical ingredient (API), which is typically included for formulation and/or administration purposes. “Diluent” and “adjuvant” are defined hereinafter.

The terms “treat”, “treating,” “treatment,” and “therapy” as used herein refer to therapy, including without limitation, curative therapy, prophylactic therapy, and preventative therapy. Prophylactic treatment generally constitutes either preventing the onset of disorders altogether or delaying the onset of a pre-clinically evident stage of disorders in individuals.

The phrase “effective amount” is intended to quantify the amount of each agent, which will achieve the goal of improvement in disorder severity and the frequency of incidence over treatment of each agent by itself, while avoiding adverse side effects typically associated with alternative therapies. The effective amount, in one embodiment, is administered in a single dosage form or in multiple dosage forms.

Deuterium (D or ²H) is a non-radioactive, stable isotope of hydrogen, the natural abundance of deuterium is 0.015%. Compound should be considered to be unnatural, if its level of deuterium has been enriched to be greater than their natural abundance level 0.015%.

In a compound of this invention, it is understood that the abundance of deuterium is substantially greater than the natural abundance of deuterium, which is 0.015%, when a particular position is designated as deuterium. A position designated as deuterium typically has a minimum isotopic enrichment factor of at least 3000 at each atom designated as deuterium in said compound. The concentration of naturally abundant stable hydrogen is small and immaterial compared to the degree of stable isotopic substitution of compounds of this invention.

In some embodiments, a compound of Formulas I-III has abundance for each designated deuterium atom of at least greater than the natural abundance of deuterium, which is 0.015%. In certain embodiments, the deuterium enrichment in compounds of Formulas I-III is at least about 1%.

In other embodiments, a compound of this invention has an isotopic enrichment factor for each designated deuterium atom of at least 3500, at least 4000, at least 4500, at least 5000, or at least 5500, at least 6000, at least 6333.3, at least 6466.7, or at least 6633.3.

The term “isotopic enrichment factor” as used herein means the ratio between the isotopic abundance and the natural abundance of a specified isotope.

Regardless of the route of administration selected, the compounds of the present invention, which may be used in a suitable hydrated form, and/or the pharmaceutical compositions of the present invention, are formulated into pharmaceutically acceptable dosage forms or by other conventional methods known to those of skill in the art.

Actual dosage levels of the active ingredients in the pharmaceutical compositions of the present invention may be varied so as to obtain an effective amount of the active ingredient to achieve the desired therapeutic response for a particular patient, composition, and mode of administration, without being toxic to the patient.

The selected dosage level will depend upon a variety of factors including the activity of the particular compound of the present invention employed, the route of administration, the time of administration, the rate of excretion of the particular compound being employed, the duration of the treatment, other drugs, compounds and/or materials used in combination with the particular PARP inhibitors employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous, intracerebroventricular and subcutaneous doses of the compounds of this invention for a patient will range from about 0.0001 to about 100 mg per kilogram of body weight per day. The mode of administration can have a large effect on dosage. Higher doses may be used for localized routes of delivery.

If desired, the effective daily dose of the active compound may be administered as two, three, four, five, six or more sub-doses administered separately at appropriate intervals throughout the day, optionally, in unit dosage forms. Those of skill in the art will readily appreciate that dose levels can vary as a function of the specific compound, the severity of the symptoms and the susceptibility of the subject to side effects. Dosages for a given compound disclosed herein are readily determinable by those of skill in the art by a variety of means.

The carbon-hydrogen bonds of all compounds contain a naturally occurring distribution of hydrogen isotopes, namely ¹H or protium (about 99.9844%), ²H or deuterium (about 0.0156%), and ³H or tritium (in the range between about 0.5 and 67 tritium atoms per 10¹⁸ protium atoms).

Increased levels of deuterium incorporation produce a detectable Kinetic Isotope Effect (KIE) that could affect the pharmacokinetic, pharmacologic and/or toxicological parameters of such anti-neoplastic agents relative to compounds having naturally occurring levels of deuterium. Some aspects of the present invention disclosed herein describe a novel approach to designing and synthesizing new analogs of these PARP inhibitors through chemical modifications and derivations of the carbon-hydrogen bonds of these PARP inhibitors and/or of the chemical precursors used to synthesize said PARP inhibitors. Suitable modifications of certain carbon-hydrogen bonds into carbon-deuterium bonds, in some embodiments, generate novel PARP inhibitors with unexpected and non-obvious improvements of pharmacological, pharmacokinetic and toxicological properties in comparison to the non-isotopically enriched anti- neoplastic agents. This invention relies on the judicious and successful application of chemical kinetics to drug design. Deuterium incorporation levels in the compounds of the invention are significantly higher than the naturally-occurring levels and are sufficient to induce at least one substantial improvement as described herein.

Various deuteration patterns are used to a) reduce or eliminate unwanted metabolites, b) increase the half-life of the parent drug, and/or c) decrease the production of deleterious metabolites in specific tissues and create a more effective drug and a safer drug for polypharmacy, whether the polypharmacy be intentional or not. The deuteration approach has strong potential to slow the metabolism via various oxidative mechanisms.

The deuterated analogs of this invention uniquely maintain the beneficial aspects of the non-isotopically enriched drugs while substantially increasing the maximum tolerated dose, decreasing toxicity, increasing the half-life (T_{1/2}), lowering the maximum plasma concentration (Cₘₐₓ) of the minimum efficacious dose (MED), lowering the efficacious dose and thus decreasing the non-mechanism-related toxicity, and/or lowering the probability of drug-drug interactions. These drugs also have strong potential to reduce the cost-of-goods (COG) owing to the ready availability of inexpensive sources of deuterated reagents combined with previously mentioned potential for lowering the therapeutic dose.

### Pharmaceutical Compositions/Formulations

One embodiment provides a pharmaceutical composition comprising a compound of Formulas I-III, or a pharmaceutically acceptable salt, solvate, stereoisomer or tautomer thereof, and at least one pharmaceutically acceptable excipient.

In some embodiments, the present invention provides methods for inhibiting PARP. The method comprises administrating to a mammalian subject a therapeutically effective amount of at least one compound of Formulas I-III. The method comprises treating or preventing stroke, myocardial infarction, neurodegenerative diseases, ovarian cancer, breast cancer, prostate cancer, lung cancer, colorectal cancer, and melanoma.

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed., Easton, Pa.: Mack Publishing Company (1995); Hoover, John E., Remington's Pharmaceutical Sciences, Mack Publishing Co., Easton, Pennsylvania (1975); Liberman, H. A. and Lachman, L , Eds., Pharmaceutical Dosage Forms, Marcel Decker, New York, N.Y. (1980); and Pharmaceutical Dosage Forms and Drug Delivery Systems, Seventh Ed., Lippincott Williams & Wilkins (1999), herein incorporated by reference for such disclosure.

A pharmaceutical composition, as used herein, refers to a mixture of a compound of formula I with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

The pharmaceutical formulations described herein are administered to a subject by appropriate administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

All formulations for oral administration are in dosages suitable for such administration. Examples of such dosage units are tablets or capsules. In some embodiments, these contain an amount of active ingredient from about 1 to 2000 mg, advantageously from about 1 to 500 mg, and typically from about 5 to 150 mg. A suitable daily dose for a human or other mammal vary widely depending on the condition of the patient and other factors, but, once again, can be determined using routine methods and practices.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethylcellulose (HPMC), hydroxypropyl-methylcellulose phthalate, hydroxypropylmethylcellulose acetate stearate (HPMCAS), sucrose, xylitol, lactitol, mannitol, sorbitol, sodium chloride, polyethylene glycol, and the like.

Suitable disintegrants for use in the solid dosage forms described herein include, but are not limited to, natural starch such as corn starch or potato starch, a pregelatinized starch, or sodium starch glycolate, a cellulose such as methylcrystalline cellulose, methylcellulose, microcrystalline cellulose, croscarmellose, or a cross-linked cellulose, such as cross-linked sodium carboxymethylcellulose, cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a gum such as agar, guar, locust bean, Karaya, pectin, or tragacanth, sodium starch glycolate, bentonite, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethyl-cellulose, methylcellulose, hydroxypropylmethylcellulose, hydroxypropylmethylcellulose acetate stearate, hydroxyethylcellulose, hydroxypropylcellulose, ethylcellulose, and microcrystalline cellulose, microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose, glucose, dextrose, molasses, mannitol, sorbitol, xylitol, lactose, a natural or synthetic gum such as acacia, tragacanth, ghatti gum, mucilage of isapol husks, starch, polyvinylpyrrolidone, larch arabogalactan, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Binder levels of up to 70% in tablet formulations are common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinyl-pyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

### Synthesis of Compounds

The examples and preparations provided below illustrated and exemplify the compounds described herein and methods of preparing such compounds. In general, the compounds described herein may be prepared by processes known in the general chemical arts.

The compounds of the present invention can be prepared using various synthetic routes, including those schemes described below, starting from commercially available materials. Starting materials of the invention, are either known, commercially available, or can be synthesized in analogy to or according to methods that are known in the art. Many starting materials may be prepared according to known processes and, in particular, can be prepared using processes described in the examples. In synthesizing starting materials, functional groups in some cases are protected with suitable protecting groups when necessary. Functional groups may be removed according to known procedures in the art.

The protection of functional groups by protecting groups, the protecting groups themselves, and their removal reactions (commonly referred to as “deprotection”) are described, for example, in standard reference works, such as J. F. W. McOmie, Protective Groups in Organic Chemistry, Plenum Press, London and New York (1973), in T. W. Greene, Protective Groups in Organic Synthesis, Wiley, New York (1981), in The Peptides, Volume 3, E. Gross and J. Meienhofer editors, Academic Press, London and New York (1981), in Methoden der Organischen Chemie (Methods of Organic Chemistry), Houben Weyl, 4^{th} edition, Volume 15/1, Georg Thieme Verlag, Stuttgart (1974), in H.-D. Jakubke and H. Jescheit, Aminosäuren, Peptide, Proteine (Amino Acids, Peptides, Proteins), Verlag Chemie, Weinheim, Deerfield Beach, and Basel (1982), and in Jochen Lehmann, Chemie der Kohlenhydrate: Monosaccharide and Derivate (Chemistry of Carbohydrates: Monosaccharides and Derivatives), Georg Thieme Verlag, Stuttgart (1974).

All synthetic procedures described herein can be carried out under known reaction conditions, advantageously under those described herein, either in the absence or in the presence (usually) of solvents or diluents. The solvents should be inert with respect to, and should be able to dissolve, the starting materials and other reagents used. Solvents should be able to partially or wholly solubilize the reactants in the absence or presence of catalysts, condensing agents or neutralizing agents, for example ion exchangers, typically cation exchangers for example in the H⁺ form. The ability of the solvent to allow and/or influence the progress or rate of the reaction is generally dependent on the type and properties of the solvent(s), the reaction conditions including temperature, pressure, atmospheric conditions such as in an inert atmosphere under argon or nitrogen, and concentration, and of the reactants themselves.

Suitable solvents for conducting reactions to synthesize compounds of the invention include, without limitation, water; esters, including lower alkyl-lower alkanoates, e.g., ethyl acetate; ethers including aliphatic ethers, e.g., Et₂O and ethylene glycol dimethylether or cyclic ethers, e.g., THF; liquid aromatic hydrocarbons, including benzene, toluene and xylene; alcohols, including MeOH, EtOH, 1-propanol, i-PrOH, n- and t-butanol; nitriles including CH₃CN; halogenated hydrocarbons, including CH₂Cl₂, CHCl₃ and CCl₄; acid amides including DMF; sulfoxides, including DMSO; bases, including heterocyclic nitrogen bases, e.g. pyridine; carboxylic acids, including lower alkanecarboxylic acids, e.g., AcOH; inorganic acids including HCl, HBr, HF, H₂SO₄ and the like; carboxylic acid anhydrides, including lower alkane acid anhydrides, e.g., acetic anhydride; cyclic, linear, or branched hydrocarbons, including cyclohexane, hexane, pentane, isopentane and the like, and mixtures of these solvents, such as purely organic solvent combinations, or water-containing solvent combinations e.g., aqueous solutions. These solvents and solvent mixtures may also be used in “working-up” the reaction as well as in processing the reaction and/or isolating the reaction product(s), such as in chromatography.

The invention further encompasses “intermediate” compounds, including structures produced from the synthetic procedures described, whether isolated or not, prior to obtaining the finally desired compound. Structures resulting from carrying out steps from a transient starting material, structures resulting from divergence from the described method(s) at any stage, and structures forming starting materials under the reaction conditions are all “intermediates” included in the invention. Further, structures produced by using starting materials in the form of a reactive derivative or salt, or produced by a compound obtainable by means of the process according to the invention and structures resulting from processing the compounds of the invention in situ are also within the scope of the invention.

In synthesizing a compound of Formulas I, II and III according to a desired procedure, the steps in some embodiment, are performed in an order suitable to prepare the compound, including a procedure described herein or by an alternate order of steps described herein, and in one embodiment, be preceded, or followed, by additional protection/deprotection steps as necessary. In certain embodiment, the procedures are further use appropriate reaction conditions, including inert solvents, additional reagents, such as bases (e.g., LDA, DIEA, pyridine, K₂CO₃, and the like), catalysts, and salt forms of the above. The intermediates in some embodiments are isolated or carried on in situ, with or without purification. Purification methods are known in the art and include, for example, crystallization, chromatography (liquid and gas phase, and the like), extraction, distillation, trituration, reverse phase HPLC and the like. Reactions conditions such as temperature, duration, pressure, and atmosphere (inert gas, ambient) are known in the art and may be adjusted as appropriate for the reaction. Synthetic chemistry transformations and protecting group methodologies (protection and deprotection) useful in synthesizing the inhibitor compounds described herein are known in the art and include, for example, those such as described in R. Larock, Comprehensive Organic Transformations, VCH Publishers (1989); T. W. Greene and P. G. M. Wuts, Protective Groups in OrganicSynthesis, 3rd edition, John Wiley and Sons (1999); L. Fieser and M. Fieser, Fieser and Fieser's Reagents for Organic Synthesis, John Wiley and Sons (1994); A. Katritzky and A. Pozharski, Handbook of Heterocyclic Chemistry, 2nd edition (2001); M. Bodanszky, A. Bodanszky, The Practice of Peptide Synthesis, Springer-Verlag, Berlin Heidelberg (1984); J. Seyden-Penne, Reductions by the Alumino- and Borohydrides in Organic Synthesis, 2nd edition, Wiley-VCH (1997); and L. Paquette, editor, Encyclopedia of Reagents for Organic Synthesis, John Wiley and Sons (1995).

In the reactions described, it is necessary in certain embodiments to protect reactive functional groups, for example hydroxy, amino, thiol or carboxy groups, where these are desired in the final product, to avoid their unwanted participation in the reactions. Protecting groups are used to block some or all reactive moieties and prevent such groups from participating in chemical reactions until the protective group is removed. In one embodiment, each protective group is removable by a different means. Protective groups that are cleaved under totally disparate reaction conditions fulfill the requirement of differential removal. In some embodiments, protective groups are removed by acid, base, and/or hydrogenolysis.

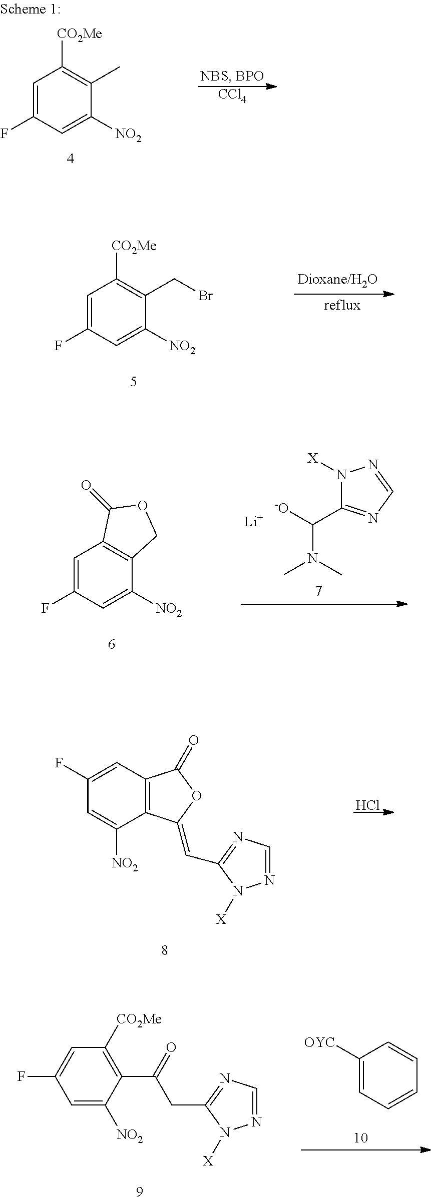

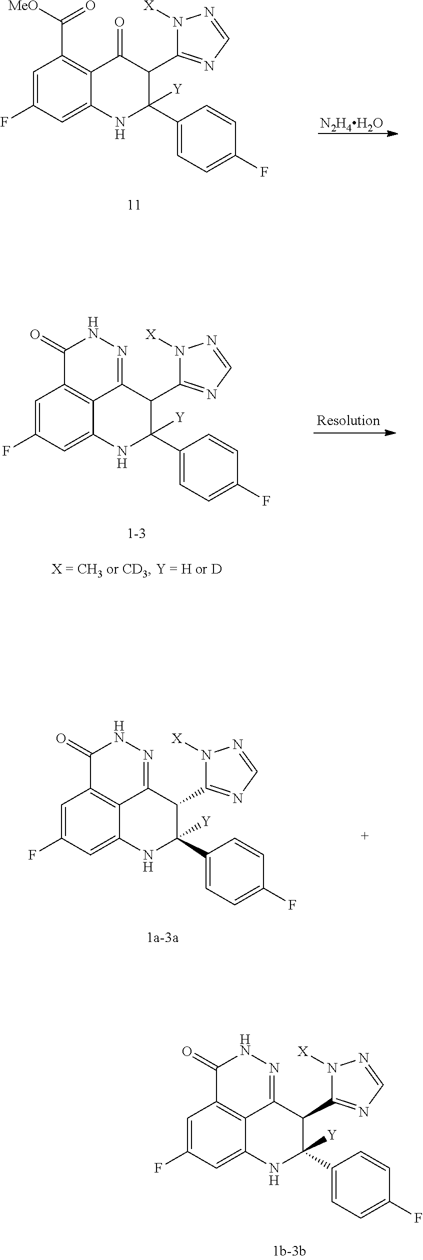

As shown in Scheme 1, 1a-3a can be obtained from the direct chiral separation of compound 1 with the preparative HPLC (or preparative Supercritical Fluid Chromatography, SFC) or indirect chemical separation (attachment of an enantiomer mixture to a chiral auxiliary, separation of the resulting mixture of diastereomers by recrystallization and liberation of an optically pure product from the auxiliary). Compound 1-3 can be prepared from the compound 6 via the aldol condensation dehydration reaction, hydrolysis of the enolate and hydrazine hydrate cyclizaion. In the aldol condensation dehydration reaction, compound 7 or its aldehyde surrogates can be used, and the solvents can be selected (not limited to) from lower boiling point ether solvent such as dimethyl ether, THF, 2-methyl-THF. In the hydrolysis reaction, compound 9 or a salt thereof can be obtained, where the inorganic acid can be HCl, acetic or trifluoroacetic acid. In the hydrazine hydrate cyclizaion, the solvent alcohol is selected (not limited to) from the group consisting of lower alkyl alcohols, such as methanol, ethanol, n-propanol, isopropanol, n-butanol, iso-butanol, tert-butanol. Compound 6 can be obtained commercially, or can be prepared from methyl 5-fluoro-2-methyl-3-nitrobenzoate by methods known in the literature or by the method described in Scheme 2.

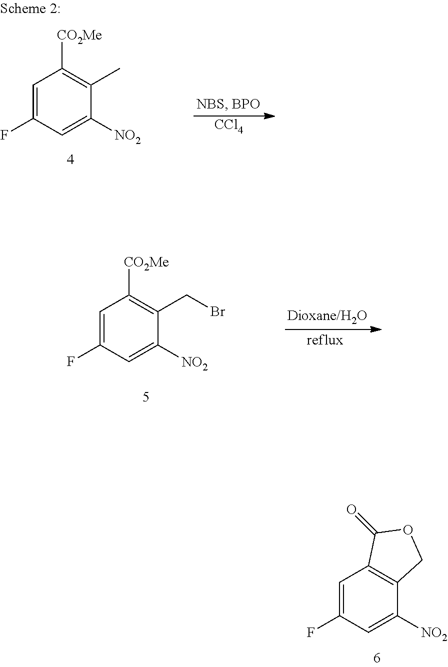 [SMILES: COC(=O)c1cc(F)cc([N+](=O)[O-])c1C ; "CCl4" ; COC(=O)c1cc(F)cc([N+](=O)[O-])c1CBr ; O=C1OCc2c1cc(F)cc2[N+](=O)[O-]]

A mixture of compound 4 methyl 5-fluoro-2-methyl-3-nitrobenzoate (3.0 g, 14 mmol, 1.0 eq., commercially available), NBS (N-Bromosuccinimide, 3.0 g, 16.8 mmol, 1.2 eq.) and BPO (dibenzoyl peroxide, 678 mg, 2.8 mmol, 0.2 eq.) in CCl₄(30 mL) was heated to reflux overnight. TLC (petroleum ether/EtOAc=5/1) showed the starting material was consumed completely. Water (20 mL) was added and the mixture was extracted with DCM (20 mL×3). The combined organic layers were washed with brine, dried over Na₂SO₄, filtered, and concentrated to give the crude compound 5, methyl 2-(bromomethyl)-5-fluoro-2-methyl-3-nitrobenzoate (4.5 g) as a light yellow oil. ¹H NMR (400 MHz, CDCl₃): δ (ppm) 7.85; (dd, J=8.0, 2.8 Hz, 1H), 7.71; (dd, J=7.2, 2.8 Hz, 1H), 5.12; (s, 2 H), 4.00; (s, 3 H).

A mixture of the crude compound 5 methyl 2-(bromomethyl)-5-fluoro-2-methyl-3-nitrobenzoate (4.5 g) in 1,4-dioxane (15 mL) and water (3 mL) was heated to reflux overnight. TLC (petroleum ether/EtOAc=5:1) showed the starting material was consumed completely. Dioxane was removed under reduced pressure. The residue was extracted with EtOAc (15 mL×4).The combined organic layers were washed with brine, dried over Na₂SO₄, concentrated to give the crude product. The crude product was purified by gel chromatography (petroleum ether to petroleum ether/EtOAc=5/1) to give compound 6, 6-fluoro-4-nitroisobenzofuran (2.3 g, 83.3% yield, two steps) as a white solid. ¹H NMR (400 MHz, CDCl₃): δ (ppm) 8.25; (dd, J=7.8, 2.2 Hz, 1H), 7.97; (dd, J=6.0, 2.0 Hz, 1H), 5.12; (d,J=2.0 Hz, 2 H).

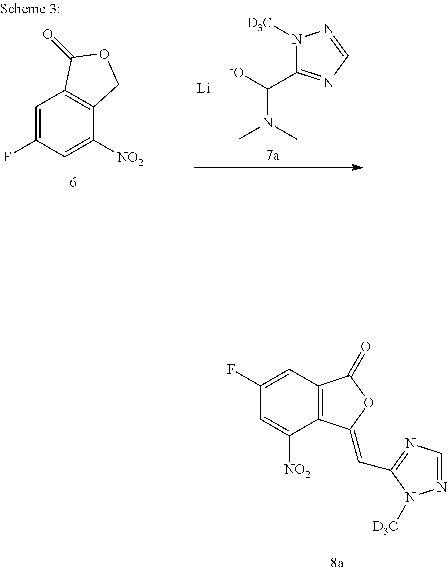

To a mixture of compound 6 (500 mg,2.54 mmol, 1.0 eq.) and compound 7a (535 mg, 3.24 mmol, 1.3 eq. prepared in Scheme 4) in anhydrous 2-Me-THF (15 mL) under inert atmosphere was added Ac₂O (1.75 mL,18.52 mmol, 7.3 eq.) dropwise. The mixture was heated to 45° C., then Et₃N (0.46 mL, 3.3 mmol) was added into the mixture. And the mixture was stirred at this temperature for additional 5 hours. After that TLC (petroleum ether/EtOAc=5/1) showed the starting material was consumed completely, the mixture was cooled to 20° C. and water (10 mL) was added dropwise. The product was collected by filtered and washed with 2-Me-THF (2 mL). The precipitate was dried in vacuo to give compound 8a (340 mg, 45.7% yield) as a yellow solid. ¹HNMR (400 MHz, d⁶-DMSO): δ (ppm) 8.60; (dd, J=8.8, 2.0 Hz, 1H), 8.43; (dd, J=6.4, 2.4 Hz, 1H), 8.11; (s, 1 H), 7.16; (s, 1 H). MS: 294; (M+H⁺).

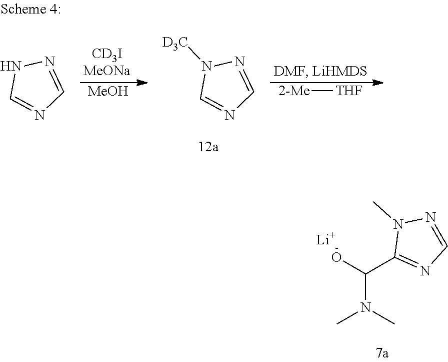

The compound 7a can be synthesized according to the route of the above scheme (Scheme 4). To a mixture of 1H-1,2,4-triazole (1.0 g, 14.48 mmol, 1.0 eq.) in MeOH (30 mL) was added MeONa (1.17 g , 21.72 mmol, 1.5 eq.). The mixture was stirred at room temperature for 30 minutes, then heated to 45° C. CD₃I (6.30 g, 43.44 mmol, 3.0 eq.) was added dropwise into the above reaction mixture at room temperature. The reaction mixture was stirred for additional 12 hours at room temperature. After that TLC (DCM/MeOH=10/1) showed the starting material was consumed completely, the mixture was concentrated in vacuo to give the crude product. The crude product was purified by gel chromatography (DCM/MeOH=20/1) to give compound 12a (420 mg, 33.7% yield) as a colorless oil.

The suspension of compound 12a (420 mg, 4.88 mmol, 1.0 eq.), 2-methyl-THF (10 mL) and DMF (0.5 mL) was cooled to an internal temperature of about 0° C. Then LiHMDS (5.86 mL, 1.0 M in THF) was added dropwise. During the addition of the LiHMDS, the desired compound was 7a was precipitated as the 2-methyl-THF or THF solvate. Then the mixture was cooled to about −30 ° C. and stirred for about 30 minutes at an internal temperature of about 0° C. The precipitate crystals were removed from the reaction mixture by filtration and washed with 2-methyl-THF. The product, compound 7a as the 2-methyl-THF solvate was dried under vacuum to give compound 7a (760 mg).

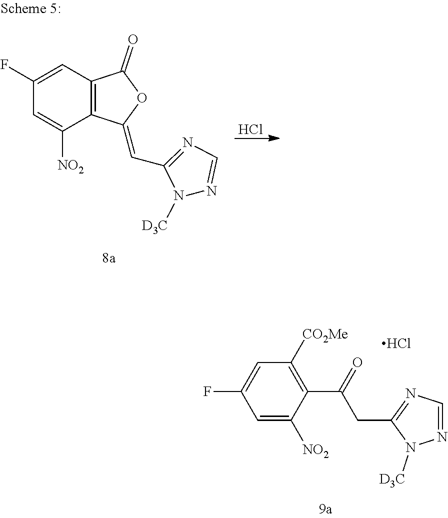 [SMILES: [2H]C([2H])([2H])n1ncnc1/C=C1\OC(=O)c2cc(F)cc([N+](=O)[O-])c21 ; Cl ; [2H]C([2H])([2H])n1ncnc1CC(=O)c1c(C(=O)OC)cc(F)cc1[N+](=O)[O-] ; Cl]

A mixture of compound 8a (340 mg, 1.16 mmol, 1.0eq.) and HCl (10 mL, 2 N in methanol) was stirred under inert atmosphere at room temperature overnight. TLC (DCM/MeOH=20/1) showed the starting material was consumed completely. Then the reaction mixture was concentrated under vacuum to give the crude compound 9a in the form of its hydrochloride salt (420 mg) as a yellow solid. ¹HNMR (400 MHz, d⁶-DMSO): δ (ppm) 8.55; (dd, J=8.4, 2.4 Hz, 1H), 8.29; (dd, J=8.2, 2.6 Hz, 1H), 8.21; (s, 1 H), 4.67; (s, 2 H), 3.91; (s, 3 H). MS: 326 (M+H⁺),

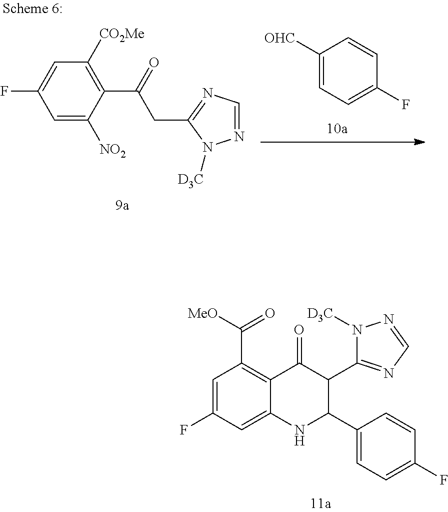 [SMILES: [2H]C([2H])([2H])n1ncnc1CC(=O)c1c(C(=O)OC)cc(F)cc1[N+](=O)[O-] ; O=Cc1ccc(F)cc1 ; [2H]C([2H])([2H])n1ncnc1C1C(=O)c2c(cc(F)cc2C(=O)OC)NC1c1ccc(F)cc1]

To a suspension of compound 9a (420 mg,1.16 mmol, 1.0eq.) and compound 10a 4-fluorobenzaldehyde (269 mg, 2.17 mmol, 1.87 eq., commercially available) in a mixture of solvents THF (18 mL) and MeOH (3 mL) was added titanium(III) chloride (6 mL, 20% w/w solution in 2N hydrochloric acid) with stirring at room temperature. The mixture was allowed to stir at 40° C. for 2 hours. Then the mixture was diluted with water (150 mL), and the resulting solution was extracted with ethyl acetate (80 mL×4). The combined organic layers were washed with saturated NaHCO₃ (50 mL×3) and aqueous NaHSO₃ (50 mL×3), dried over Na₂SO₄, and concentrated to dryness. The crude solid was purified by gel chromatography (DCM/MeOH=80/1) to give the title compound 11a (350 mg) compound as a yellow oil. MS: 402 (M+H⁺).

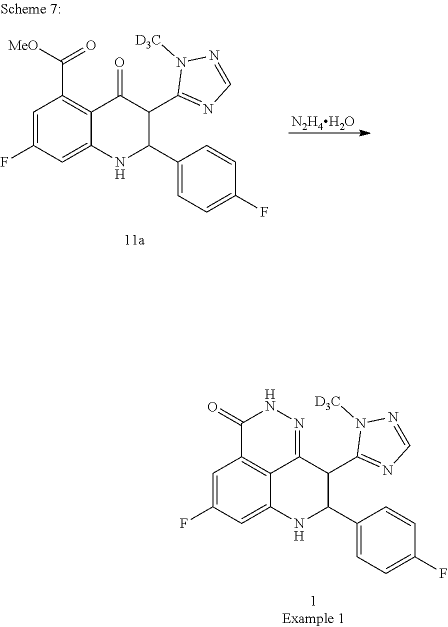 [SMILES: [2H]C([2H])([2H])n1ncnc1C1C(=O)c2c(cc(F)cc2C(=O)OC)NC1c1ccc(F)cc1 ; "N2H4·H2O" ; [2H]C([2H])([2H])n1ncnc1C1c2n[nH]c(=O)c3cc(F)cc(c23)NC1c1ccc(F)cc1]

The suspension of compound 11a (350 mg, 0.87 mmol, 1.0 eq.) in methanol (5 mL) was stirred at room temperature for 15 minutes. The hydrazine hydrate (3 mL) was added dropwise into the above reaction mixture at ambient temperature. Then the reaction mixture was stirred at room temperature overnight. TLC (DCM/MeOH=20/1) showed the starting material was consumed completely. The obtained slurry was filtered. The wet cake was suspended in methanol (2 mL) and stirred at room temperature for 3 hours. The above slurry was filtered, and the wet cake was washed with methanol. Then the wet cake was dried to give the title compound 1 (Example 1) as a white solid (172 mg, 51.6% yield). ¹HNMR (400 MHz, d⁶-DMSO): δ (ppm) 12.35; (s, 1 H), 7.80; (s, 1 H), 7.72; (s, 1 H), 7.49; (dd, J=8.6, 5.4 Hz, 2H), 7.16; (t, J=8.8 Hz, 2H), 7.07; (dd, J=8.8, 2.4 Hz, 1H), 6.92; (dd, J=11.2, 2.4 Hz, 1H), 5.00; (m, 2H). MS: 384; (M+H⁺).

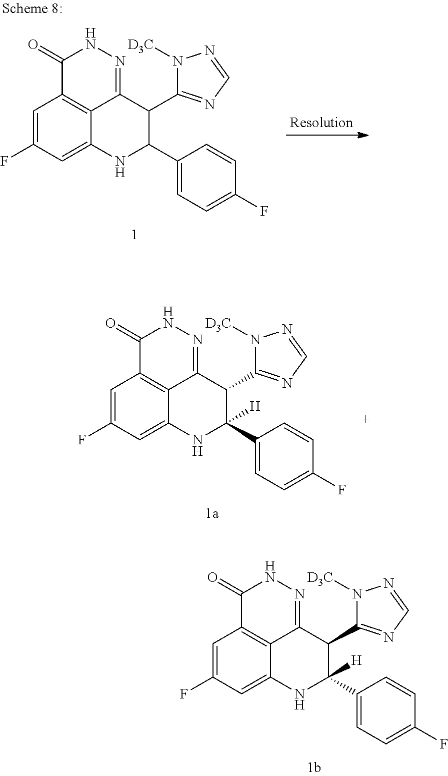 [SMILES: [2H]C([2H])([2H])n1ncnc1C1c2n[nH]c(=O)c3cc(F)cc(c23)NC1c1ccc(F)cc1 ; [2H]C([2H])([2H])n1ncnc1[C@H]1c2n[nH]c(=O)c3cc(F)cc(c23)N[C@@H]1c1ccc(F)cc1 ; [2H]C([2H])([2H])n1ncnc1[C@@H]1c2n[nH]c(=O)c3cc(F)cc(c23)N[C@H]1c1ccc(F)cc1]

A chiral resolution of compound 1 (246.6 mg, 99.2%) was carried out on preparative HPLC using a ChiralPak IG column and DCM/methnaol (v/v : 90/10) as a mobile phase. This afforded two enantiomers with retention times of 4.403 minute (100 mg, recovery 81.1%, >99% ee) and 4.976 minute (120 mg, recovery 97.3%, >99% ee).

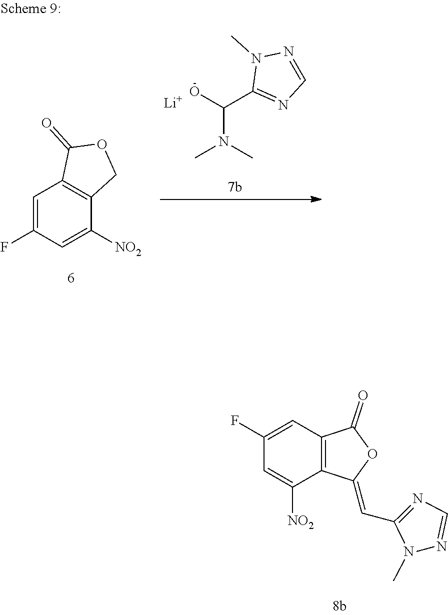 [SMILES: O=C1OCc2c1cc(F)cc2[N+](=O)[O-] ; [Li+] ; CN(C)C(O)c1ncnn1C ; Cn1ncnc1/C=C1\OC(=O)c2cc(F)cc([N+](=O)[O-])c21]

The compound 8b was synthesized in a manner similar to preparation of 8a (Scheme 3). Compound 8b was obtained in 47.5% yield starting from 500 mg of compound 6. ¹HNMR (400 MHz, d⁶-DMSO): δ (ppm) 8.61; (dd, J=8.6, 2.2 Hz, 1H), 8.43; (dd, J=6.4, 2.4 Hz, 1H), 8.11; (s, 1 H), 7.17; (s, 1 H) , 3.95; (s, 3 H). MS: 291; (M+H⁺).

The compound 7b can be synthesized according to the preparation of 7a (Scheme 4). Compound 7b was obtained in 90.9% yield starting from 480 mg of 1-methyl-1H-1,2,4-triazole.

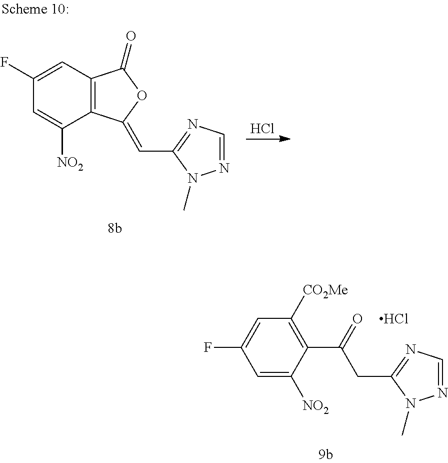 [SMILES: Cn1ncnc1/C=C1\OC(=O)c2cc(F)cc([N+](=O)[O-])c21 ; Cl ; COC(=O)c1cc(F)cc([N+](=O)[O-])c1C(=O)Cc1ncnn1C ; Cl]

The compound 9b in the form of its hydrochloride salt was synthesized in a manner similar to preparation of 9a hydrochloride salt (Scheme 5). Compound 9b hydrochloride salt was obtained in 95% yield starting from 300 mg of compound 8b. ¹HNMR (400 MHz, d⁶-DMSO): δ(ppm) 8.56; (dd, J=8.0, 2.4 Hz, 1H), 8.29; (dd, J=8.4, 2.4 Hz, 1H), 8.21; (s, 1 H), 4.71; (s, 2 H), 3.93; (s, 3 H), 3.91; (s, 3 H). MS: 323; (M+H⁺).

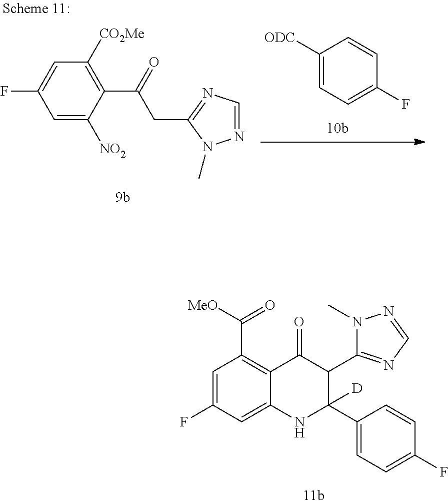

The crude compound 11b was synthesized in a manner similar to preparation of crude 11a (Scheme 6). 300 mg of compound 11b was obtained starting from 350 mg of compound 9b. MS: 400 (M+H⁺).

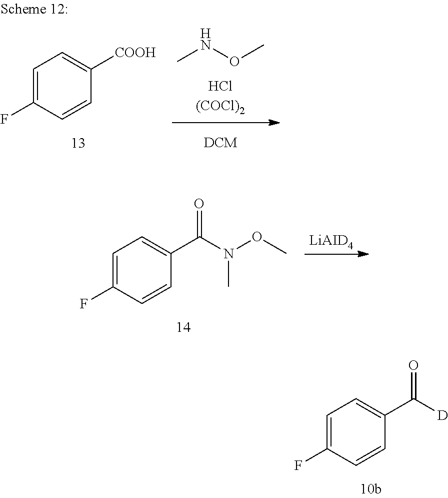

The compound 10b can be synthesized according to the route of the above scheme(Scheme 11). To a mixture of compound 13 4-fluorobenzoic acid(3 g, 21.42 mmol, 1.0 eq.) in DCM (30 mL) and DMF (0.3 mL) was added oxaloyl chloride (2.99 g , 23.55 mmol, 1.1 eq.) slowly. Then the reaction mixture was stirred at room temperature for 1 hour. After that N,O-Dimethylhydroxylamine hydrochloride (2.5 g, 25.69 mmol, 1.2 eq.) and Et₃N (9.0 mL, 62.4 mmol, 1.2 eq.) were added into the reaction mixture. And the mixture was stirred at room temperature for additional 2 hours. TLC (petroleum ether/EtOAc=10/1) showed the starting material was consumed completely. The mixture was poured into water (100 mL), extracted with EtOAc (100 mL×3). The combined organic layers were washed with brine (100 mL×1), dried over Na₂SO₄, filtered, and concentrated in vacuo to give the crude compound 14. The crude product was purified by gel chromatography (petroleum ether/EtOAc=20/1) to give compound 8b (2.1 g, 53.9% yield) as a colorless oil. MS: 184 (M+H⁺).

To a mixture of compound 14 (1.0 g, 5.46 mmol, 1.0 eq.) in THF (30 mL) was added LiAlD₄ (275 mg , 6.55 mmol, 1.2 eq.) by portion. The mixture was stirred at room temperature for 30 minutes. TLC (petroleum ether/EtOAc=10/1) showed the starting material was consumed completely. The mixture was poured into NH₄Cl (aq.) (100 mL) and extracted with EtAc. (100 mL×3). The combined organic layers were washed with brine (100 mL×1), dried over Na₂SO₄, filtered, and concentrated in vacuo to give the crude compound 10b (312 mg), which can be used to the next step directly without purification.

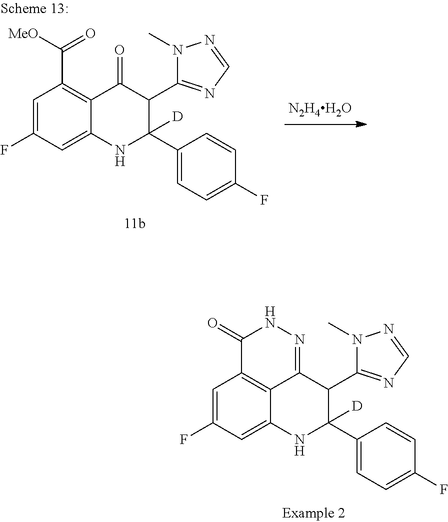 [SMILES: [2H]C1(c2ccc(F)cc2)Nc2cc(F)cc(C(=O)OC)c2C(=O)C1c1ncnn1C ; "N2H4·H2O" ; [2H]C1(c2ccc(F)cc2)Nc2cc(F)cc3c(=O)[nH]nc(c23)C1c1ncnn1C]

The compound 2 (Example 2) was synthesized in a manner similar to preparation of 1 (Scheme 7). Compound 2 was obtained in 57.7% yield starting from 300 mg of compound 11b. ¹HNMR (400 MHz, d⁶-DMSO): δ (ppm) 12.35; (s, 1 H), 7.80; (s, 1 H), 7.70; (s, 1 H), 7.49; (dd, J=8.8, 5.6 Hz, 2H), 7.16; (t, J=8.8 Hz, 2H), 7.07; (dd, J=8.8, 2.4 Hz, 1H), 6.92; (dd, J=11.2, 2.4 Hz, 1H), 5.01; (s, 1H), 3.66; (s, 3H). MS: 382; (M+H⁺).

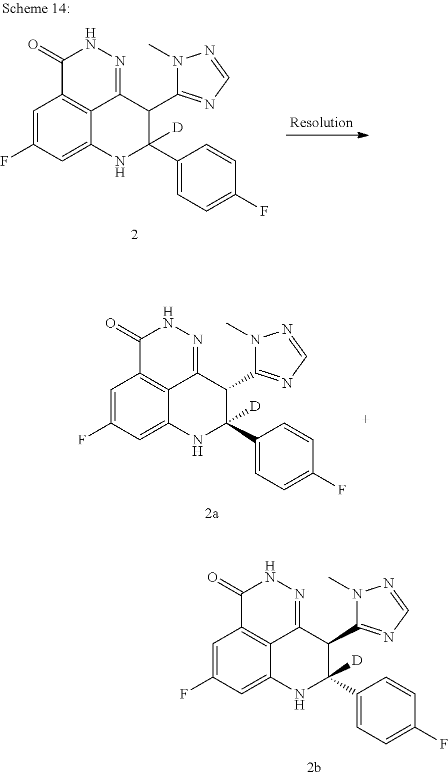 [SMILES: [2H]C1(c2ccc(F)cc2)Nc2cc(F)cc3c(=O)[nH]nc(c23)C1c1ncnn1C ; [2H][C@]1(c2ccc(F)cc2)Nc2cc(F)cc3c(=O)[nH]nc(c23)[C@@H]1c1ncnn1C ; [2H][C@@]1(c2ccc(F)cc2)Nc2cc(F)cc3c(=O)[nH]nc(c23)[C@H]1c1ncnn1C]

A chiral resolution of compound 2 (183 mg, 98.6%) was carried out on preparative HPLC using a ChiralPak IG column and DCM/methnaol (v/v : 90/10) as a mobile phase. This afforded two enantiomers with retention times of 4.413 minute (70 mg, recovery 76.5%, >99% ee) and 4.978 minute (52 mg, recovery 56.8%, >99% ee).

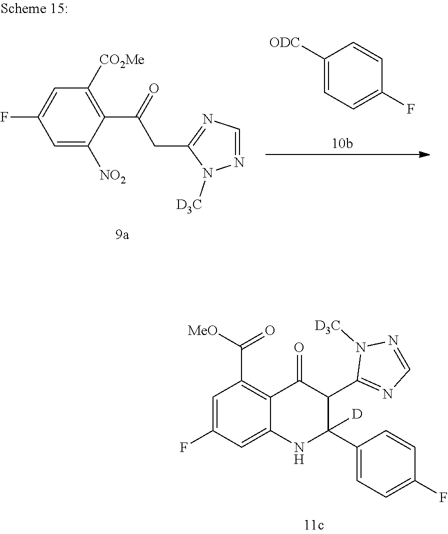

The crude compound 11c was synthesized in a manner similar to preparation of crude 11a (Scheme 6). 150 mg of compound 11c was obtained starting from 350 mg of compound 9a. MS: 403 (M+H⁺).

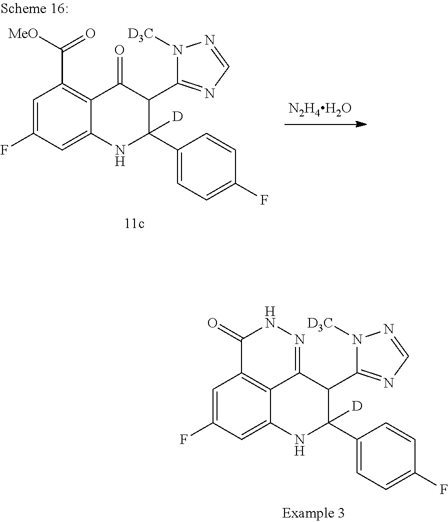 [SMILES: [2H]C1(c2ccc(F)cc2)Nc2cc(F)cc(C(=O)OC)c2C(=O)C1c1ncnn1C([2H])([2H])[2H] ; "N2H4·H2O" ; [2H]C1(c2ccc(F)cc2)Nc2cc(F)cc3c(=O)[nH]nc(c23)C1c1ncnn1C([2H])([2H])[2H]]

The compound 3 (Example 3) was synthesized in a manner similar to preparation of 1 (Scheme 7). Compound 3 was obtained in 42.2% yield starting from 150 mg of compound 11c. ¹HNMR (400 MHz, d⁶-DMSO): δ (ppm) 12.35 (s, 1 H), 7.80; (s, 1 H), 7.71; (s, 1 H), 7.49; (dd, J=8.8, 5.6 Hz, 2H), 7.16; (t, J=9.2 Hz, 2H), 7.07; (dd, J=9.2, 2.4 Hz, 1H), 6.92; (dd, J=11.2, 2.4 Hz, 1H), 5.01; (s, 1H). MS: 385; (M+H⁺).

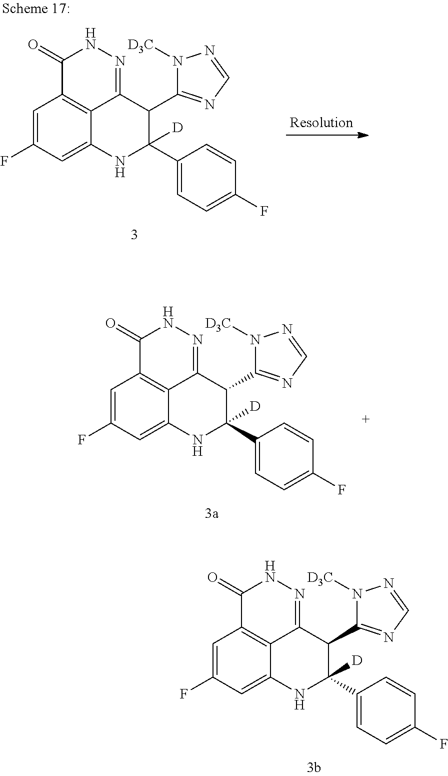 [SMILES: [2H]C1(c2ccc(F)cc2)Nc2cc(F)cc3c(=O)[nH]nc(c23)C1c1ncnn1C([2H])([2H])[2H] ; [2H]C([2H])([2H])n1ncnc1[C@H]1c2n[nH]c(=O)c3cc(F)cc(c23)N[C@]1([2H])c1ccc(F)cc1 ; [2H]C([2H])([2H])n1ncnc1[C@@H]1c2n[nH]c(=O)c3cc(F)cc(c23)N[C@@]1([2H])c1ccc(F)cc1]

A chiral resolution of compound 3 (286.7 mg) was carried out on preparative HPLC using a ChiralPak IC column and DCM/methnaol (v/v : 90/10) as a mobile phase. This afforded two enantiomers with retention times of 2.734 minute (137.0 mg, recovery 95.6%, >99% ee) and 3.252 minute (128.8 mg, recovery 89.9%, >99% ee).

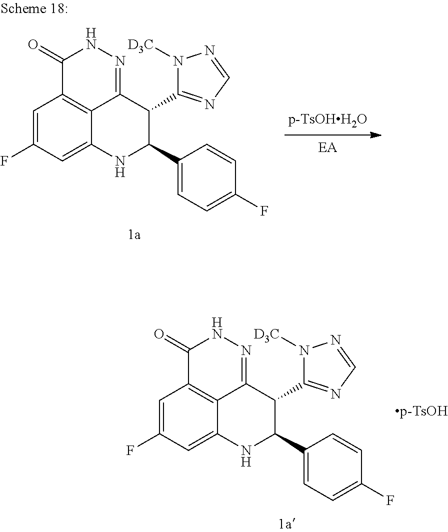 [SMILES: [2H]C([2H])([2H])n1ncnc1[C@H]1c2n[nH]c(=O)c3cc(F)cc(c23)N[C@@H]1c1ccc(F)cc1 ; [2H]C([2H])([2H])n1ncnc1[C@H]1c2n[nH]c(=O)c3cc(F)cc(c23)N[C@@H]1c1ccc(F)cc1]

The compound 1a′ can be synthesized according to the route of the above scheme (Scheme 18). The solution of p-Toluenesulfonic acid monohydrate (10.4 mg, 54.6 μmol, 1.05 eq.) in EtAc (0.5 mL) was added into the mixture of compound 1a (20 mg, 52 μmol, 1.0 eq.) in EA (6 mL) to give compound 1a′ (24 mg, 83.1% yield). ¹H NMR (400 MHz, d⁶-DMSO): δ (ppm) 12.36; (s, 1H), 7.84; (s, 1H), 7.74; (s, 1H), 7.51-7.46; (m, 4H), 7.16; (t, J=8.8 Hz, 2H), 7.11; (d, J=8.0 Hz, 2H), 7.07; (dd, J=8.8, 2.4 Hz, 1H), 6.92; (dd, J=10.4, 2.4 Hz, 1H), 5.01; (m, 2H), 2.29; (s, 3H).

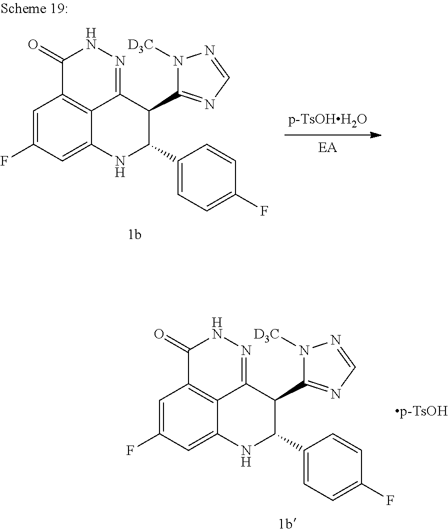 [SMILES: [2H]C([2H])([2H])n1ncnc1[C@@H]1c2n[nH]c(=O)c3cc(F)cc(c23)N[C@H]1c1ccc(F)cc1 ; [2H]C([2H])([2H])n1ncnc1[C@@H]1c2n[nH]c(=O)c3cc(F)cc(c23)N[C@H]1c1ccc(F)cc1]

The compound 1b′ was synthesized in a manner similar to preparation of 1a′ (Scheme 18). Compound 1b′ (23 mg) was obtained in 79.6% yield starting from 20 mg of compound 1b. ¹H NMR (400 MHz, d⁶-DMSO): δ (ppm) 12.37; (s, 1H), 7.84; (s, 1H), 7.74; (s, 1H), 7.52-7.44; (m, 4H), 7.16; (t, J=8.8 Hz, 2H), 7.07; (d, J=7.6 Hz, 2H), 7.07; (dd, J=9.2, 2.4 Hz, 1H), 6.92; (dd, J=11.2, 2.4 Hz, 1H), 5.01; (m, 2H), 2.29; (s, 3H).

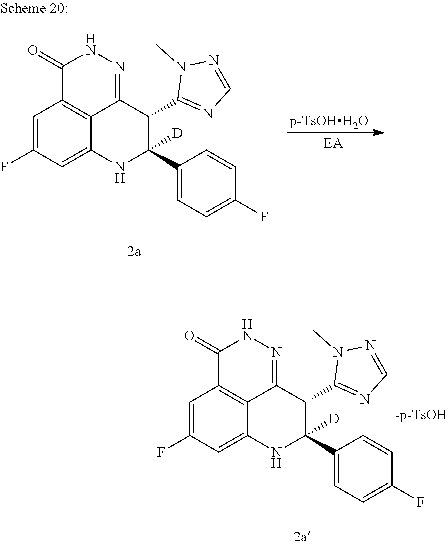 [SMILES: [2H][C@]1(c2ccc(F)cc2)Nc2cc(F)cc3c(=O)[nH]nc(c23)[C@@H]1c1ncnn1C ; [2H][C@]1(c2ccc(F)cc2)Nc2cc(F)cc3c(=O)[nH]nc(c23)[C@@H]1c1ncnn1C]

The compound 2a′ was synthesized in a manner similar to preparation of 1a′ (Scheme 18). Compound 2a′ (20 mg) was obtained in 69.5% yield starting from 20 mg of compound 2a. ¹H NMR (400 MHz, d⁶-DMSO): δ (ppm) 12.36; (s, 1H), 7.83; (s, 1H), 7.72; (s, 1H), 7.52-7.44; (m, 4H), 7.16; (t, J=8.8 Hz, 2H), 7.11; (d, J=8.0 Hz, 2H), 7.07; (dd, J=8.8, 2.4 Hz, 1H), 6.92; (dd, J=11.2, 2.4 Hz, 1H), 5.03; (s, 1H), 3.66; (s, 3H), 2.29; (s, 3H).

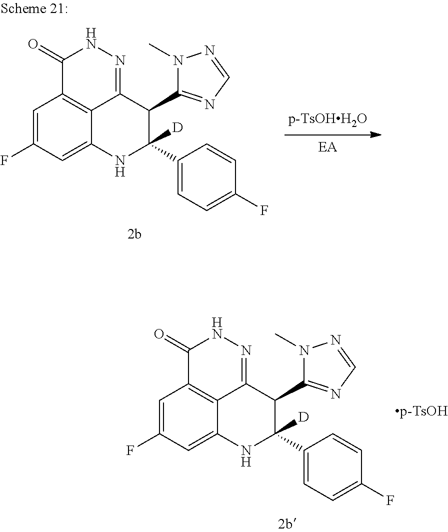 [SMILES: [2H][C@@]1(c2ccc(F)cc2)Nc2cc(F)cc3c(=O)[nH]nc(c23)[C@H]1c1ncnn1C ; [2H][C@@]1(c2ccc(F)cc2)Nc2cc(F)cc3c(=O)[nH]nc(c23)[C@H]1c1ncnn1C]

The compound 2b′ was synthesized in a manner similar to preparation of 1a′ (Scheme 18). Compound 2a′ (22 mg) was obtained in 76.4% yield starting from 20 mg of compound 2b. ¹H NMR (400 MHz, d⁶-DMSO): δ (ppm) 12.36; (s, 1H), 7.84; (s, 1H), 7.72; (s, 1H), 7.52-7.45; (m, 4H), 7.16; (t, J=8.8 Hz, 2H), 7.11; (d, J=8.0 Hz, 2H), 7.07; (dd, J=9.2, 2.4 Hz, 1H), 6.92; (dd, J=11.2, 2.4 Hz, 1H), 5.03; (s, 1H), 3.66; (s, 3H), 2.29; (s, 3H).

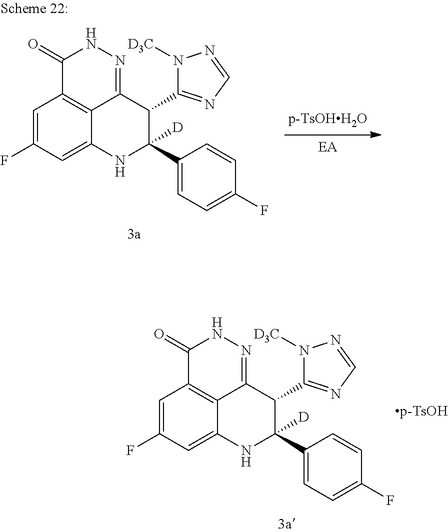 [SMILES: [2H]C([2H])([2H])n1ncnc1[C@H]1c2n[nH]c(=O)c3cc(F)cc(c23)N[C@]1([2H])c1ccc(F)cc1 ; [2H]C([2H])([2H])n1ncnc1[C@H]1c2n[nH]c(=O)c3cc(F)cc(c23)N[C@]1([2H])c1ccc(F)cc1]

The compound 3a′ was synthesized in a manner similar to preparation of 1a′ (Scheme 18). Compound 3a′ (25 mg) was obtained in 86.5% yield starting from 20 mg of compound 3a. ¹H NMR (400 MHz, d⁶-DMSO) δ (ppm) 12.36 (s, 1H), 7.84; (s, 1H), 7.72; (s, 1H), 7.52-7.45; (m, 4H), 7.16; (t, J=8.8 Hz, 2H), 7.11; (d, J=8.0 Hz, 2H), 7.07; (dd, J=9.2, 2.4 Hz, 1H), 6.92; (dd, J=11.2, 2.3 Hz, 1H), 5.03; (s, 1H), 2.29; (s, 3H).

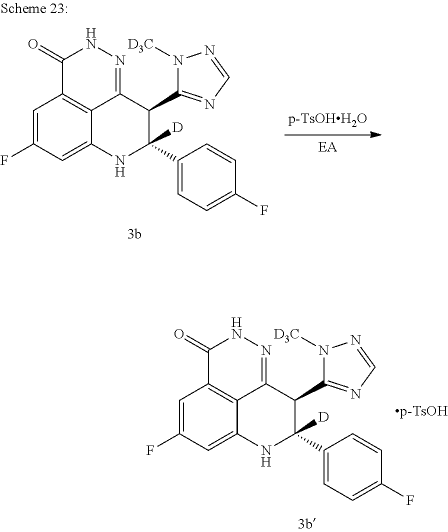 [SMILES: [2H]C([2H])([2H])n1ncnc1[C@@H]1c2n[nH]c(=O)c3cc(F)cc(c23)N[C@@]1([2H])c1ccc(F)cc1 ; [2H]C([2H])([2H])n1ncnc1[C@@H]1c2n[nH]c(=O)c3cc(F)cc(c23)N[C@@]1([2H])c1ccc(F)cc1]

The compound 3b′ was synthesized in a manner similar to preparation of 1a′ (Scheme 18). Compound 3b′ (21 mg) was obtained in 72.6% yield starting from 20 mg of compound 3b. ¹H NMR (400 MHz, d⁶-DMSO): δ (ppm) 12.36; (s, 1H), 7.84; (s, 1H), 7.72; (s, 1H), 7.53-7.44; (m, 4H), 7.16; (t, J=8.8 Hz, 2H), 7.11; (d, J=8.0 Hz, 2H), 7.07; (dd, J=8.8, 2.0 Hz, 1H), 6.92; (dd, J=11.2, 2.4 Hz, 1H), 5.03; (s, 1H), 2.29; (s, 3H).

### Biological Evaluation

PARP-1 enzymtic activity can be measured using a commercial 96-well colorimetric assay kit (4676-096-K, Trevigen, Inc). PARP-1 catalyzes the NAD-dependent addition of poly(ADP-ribose) to its nuclear protein substrates such as histones. The assay kit measures the incorporation of biotinylated Poly(ADP-ribose) onto histone proteins in a 96-well format.

Reference compound and test compounds are serially diluted with a 1× buffer. To each well of histone pre-coated plate 10 μl of 5-fold concentrations of testing compounds or reference compound, 15 μl of PARP-1 enzyme (0.5 unit) and 25 μl reaction buffer are added and the plates are incubated at room temperature for 60 min. The plates are washed with 200 μl PBS with 0.1% Triton X-100 twice and then with 200 μl PBS twice. The residual liquid is removed by carefully tapping the plates on paper towels. Equal volumes of PeroxyGlow™ solution A and B are mixed and 100 μl of the solution is added to each well. The luminescence readings are read immediately in a Synergy H1 Hybird reader (BIOTEK). The obtained luminescence readings are analyzed using a commercial graphic software (GraphPad Prism 5) and plotted against the Log scale of the compound concentrations. The IC₅₀ values are obtained by fitting the data points with the equation of Y (Luminescence reading)=minimal luminescence reading+(maximal luminescence reading−minimal luminescence reading)/(1+10̂(LogC−LogEC₅₀)), where C is the concentration of the testing compound.

The cytotoxic or cytostatic activity of Formulas I-III exemplary compounds can be measured by BRCA deficient tumor cell lines such as CAPAN-1 in a cell culture medium, adding a test compound, culturing the cells for a period of 5 days by measuring cell viability via MTT assays. Dose response data are obtained for each test compound and the degree of inhibition of tumor cell growth is expressed as an IC₅₀ value. BRCA deficient cancer cells are known to be sensitive to PARP inhibition.

Compared to Talazoparib, Formula I-III exemplary compounds may have better metabolic stability, therefore, better pharmacokinetic profile. Compared to Talazoparib, example compounds 1, 2 or 3 (the deuterated Talazoparib) may have better metabolic stability, therefore, better pharmacokinetic profile.

1. A compound of Formula I: 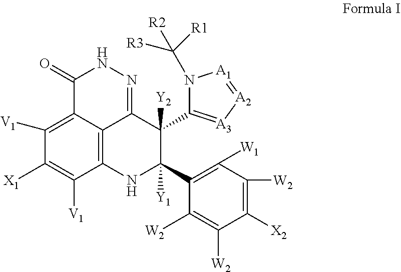
or a pharmaceutically acceptable salt, solvate, stereoisomer or tautomer thereof, wherein
R₁, R₂, R₃, Y₁, Y₂, V₁, W₁, W₂, X₁, and X₂ are independently selected from H, deuterium and F, provided that R₁, R₂, R₃, V₁, W₁, W₂, Y₁, Y₂, X₁, and X₂ contain at least one deuterium;
A₁, A₂, and A₃ are independently selected from N and CH.

2. The compound of claim 1, wherein X₁ is F.

3. The compound of claim 1, wherein X₂ is F.

4. The compound of claim 1, wherein X₁ and X₂ are both F.

5. The compound of claim 1, wherein A₁ is N, A₂ is CH and A₃ is N.

6. The compound of claim 5, wherein Y₁ is deuterium.

7. The compound of claim 5, wherein —CR₁R₂R₃ is —CD₃.

8. The compound of claim 5, wherein Y₂ is deuterium.

9. A compound of Formula II: 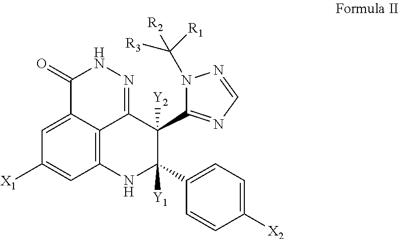
or a pharmaceutically acceptable salt, solvate, stereoisomer or tautomer thereof, wherein
R₁, R₂, R₃, Y₁, Y₂, X₁, and X₂ are independently selected from H, deuterium and F, provided that R₁, R₂, R₃, Y₁, Y₂, X₁, and X₂ contain at least one deuterium.

10. The compound of claim 9, wherein X₁ is F.

11. The compound of claim 9, wherein X₂ is F.

12. The compound of claim 9, wherein X₁ and X₂ are both F.

13. The compound of claim 12, wherein Y₁ is deuterium.

14. The compound of claim 12, wherein —CR₁R₂R₃ is —CD₃.

15. The compound of claim 12, wherein Y₂ is deuterium.

16. A compound of Formula III: 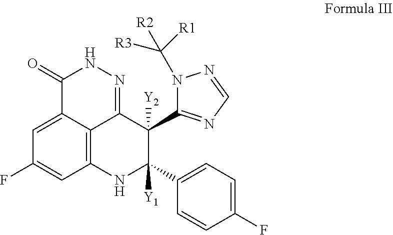
or a pharmaceutically acceptable salt, solvate, stereoisomer or tautomer thereof, wherein
R₁, R₂, R₃, Y₁, and Y₂ are independently selected from H, deuterium and F, provided that R₁, R₂, R₃, Y₁, and Y₂ contain at least one deuterium.

17. The compound of claim 16, wherein Y₁ is deuterium.

18. The compound of claim 16, wherein —CR₁R₂R₃ is —CD₃.

19. The compound of claims 1, wherein the compound is selected from the group consisting of:

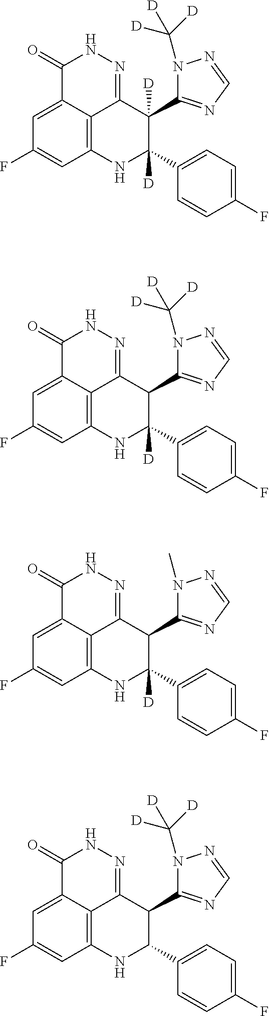 [SMILES: [2H]C([2H])([2H])n1ncnc1[C@]1([2H])c2n[nH]c(=O)c3cc(F)cc(c23)N[C@@]1([2H])c1ccc(F)cc1 ; [2H]C([2H])([2H])n1ncnc1[C@@H]1c2n[nH]c(=O)c3cc(F)cc(c23)N[C@@]1([2H])c1ccc(F)cc1 ; [2H][C@@]1(c2ccc(F)cc2)Nc2cc(F)cc3c(=O)[nH]nc(c23)[C@H]1c1ncnn1C ; [2H]C([2H])([2H])n1ncnc1[C@@H]1c2n[nH]c(=O)c3cc(F)cc(c23)N[C@H]1c1ccc(F)cc1]

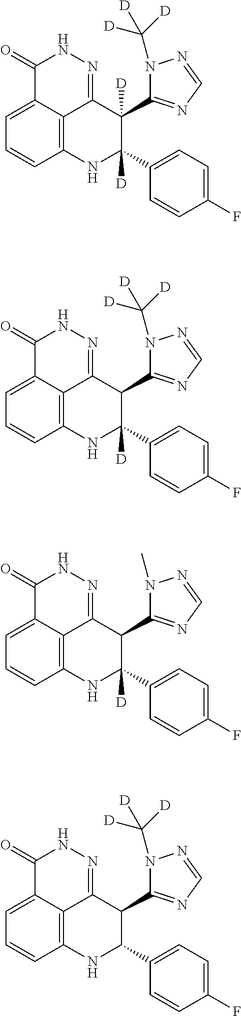 [SMILES: [2H]C([2H])([2H])n1ncnc1[C@]1([2H])c2n[nH]c(=O)c3cccc(c23)N[C@@]1([2H])c1ccc(F)cc1 ; [2H]C([2H])([2H])n1ncnc1[C@@H]1c2n[nH]c(=O)c3cccc(c23)N[C@@]1([2H])c1ccc(F)cc1 ; [2H][C@@]1(c2ccc(F)cc2)Nc2cccc3c(=O)[nH]nc(c23)[C@H]1c1ncnn1C ; [2H]C([2H])([2H])n1ncnc1[C@@H]1c2n[nH]c(=O)c3cccc(c23)N[C@H]1c1ccc(F)cc1]

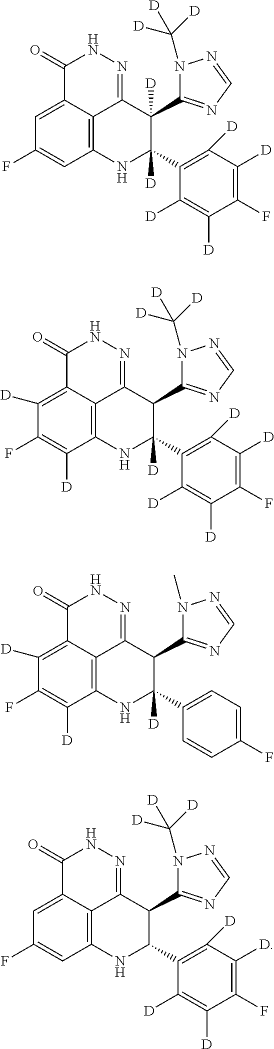

20-22. (canceled)

23. A method for treating a disease or disorder related to PARP inhibition, comprising administering a compound according to claim 1, wherein the disorder is related hyperplasia related to defective DNA repair pathways, BRCA1 and/or BRCA2 mutations, or hyperplasia.

24-26. (canceled)
